(19) United States
(12) Patent Application Publication
(10) Pub. No.: US 2014/0378284 A1
(43) Pub. Date: Dec. 25, 2014

(54) METHOD FOR ANALYZING THE SOFTNESS OF A SHEET OF TISSUE PAPER

(71) Applicant: SCA TISSUE FRANCE, Saint-Ouen (FR)

(72) Inventors: Gilbert Schohn, Saint-Hyppolyte (FR); Hubert Pfister, Rorschwihr (FR); Corinne Munerelle, Bergheim (FR)

(73) Assignee: SCA TISSUE FRANCE, Saint-Quen (FR)

(21) Appl. No.: 14/374,539

(22) PCT Filed: Jan. 30, 2013
(86) PCT No.: PCT/IB2013/000102

§ 371 Date: Jul. 25, 2014

(30) Foreign Application Priority Data
Jan. 31, 2012 (FR) 12 50885

(51) Int. Cl. G01N 33/34; G06F 17/18; B31D 1/00; G01N 21/35

(52) U.S. Cl. CPC G01N 33/346; G01N 21/3563; G01N 21/359; G06F 17/18; B31D 1/00; G01N 2201/129; G01N 2033/0078
USPC 493/8; 250/339.01; 250/339.07

(57) ABSTRACT

A method for determining the softness of a sheet of tissue paper through NIR spectrometry, includes, after having created a softness values model according to data obtained through NIR spectrometry for a set of reference tissue paper sheets, carrying out the spectral analysis of said sheet and determining the softness value on the basis of the model. The method includes obtaining the softness values of the reference tissue paper sheets of the model through sensory analysis.

### CROSS-REFERENCE TO PRIOR APPLICATION

This application is a §371 National Stage Application of PCT International Application No. PCT/IB2013/000102 filed on Jan. 30, 2013, which claims priority to French Patent Application No. 12 50885 filed on Jan. 31, 2012, both of which are incorporated herein in their entirety.

### BACKGROUND

This analysis technique is based on the principle of absorption of a light beam in the near infrared (NIR) domain by the organic material. It includes sending an infrared light signal onto the object to be analysed and comparing the signal reflected by the material or transmitted through it with the transmitted signal. The modifications of the signal give a characteristic spectrum which is interpreted using chemometrics.

The technique of infrared spectroscopy in the near infrared domain is thus known per se for identifying and quantifying the chemical components contained in a product, such as paper, through recording and analysis of their spectrum. The components can be identified, on the one hand, on the basis of their specific absorption of the beam, and can be quantified, on the other hand, on the basis of the intensity of said beam.

As well as the components, this technique also allows the physical properties of a paper to be evaluated. For example, the patent U.S. Pat. No. 6,476,915 describes a dynamic method for measuring a plurality of characteristic properties of the paper on a moving sheet, such as during production, in which optical spectra contained in the infrared (IR) range are implemented and are evaluated using a chemometric method, knowing that a correlation exists between the form of the spectrum and the parameters of the sheet. Initially, basic properties of the paper, such as grammage, wetness and thickness, are determined in a first step. Other properties are then determined using a three-level modeling. In said modeling, determinations of the composition of the paper at a first level, of the freeness index with mechanical tearing and bursting strengths, and the elasticity coefficient notably at a second modeling layer, and of individual quantities, such as its optical properties, at a third layer are successively carried out.

The patent EP 0 759 160 also describes a method aiming to quantify physical properties of a paper treated with chemical products. Firstly, it comprises the development of a calibration model through analysis of spectral data of absorption, reflection or transmission of samples of paper of which the physical properties are known. This analysis is carried out through the application of chemometric techniques. The model is then applied to the values measured through spectrometry on the samples of which the physical properties are sought to be known. The physical properties specified in this document are wet strength, dry tensile strength, wettability and others.

The properties which the paper manufacturer is drawn to analyse, and for which he seeks to improve the analysis methods, include softness, as this is an important criterion to the consumer of paper products for sanitary or domestic usage who wishes to evaluate the quality thereof.

Softness can be defined as being a sensory tactile response of a texture pleasant to touch and hold in the hand. It can also correspond to the feel of a delicate texture which presents no stiffness.

Softness is therefore often defined by its two main components: surface softness and volume softness, referred to as “textile feel”. Surface softness is the softness perceived by the end of the fingers; it depends on the surface condition and fineness of the paper. The textile feel is the softness perceived when the paper is held in the hand by rolling it into a ball; it depends on the rigidity and capacity of the fibres to move in the structure. The combined sensory response of these two types is the physical measurement of softness. This measurement is normally evaluated by a group or panel of persons, representing users of the product; it is therefore subjective. It is the result of a comparison by the persons on the panel of the tested products with a reference product.

Along with these sensory analysis methods, objective methods performing measurements of physical parameters have been developed. The Tappi method, for example, uses a device for measuring the force required to pass a sample through a calibrated gap. An indication of the feel, softness and drape are obtained by this method.

A different method determines softness on the basis of the measurement of the rigidity of a sheet of paper. The Kawabata method is also known, which measures a plurality of parameters. A different method for measuring softness which is carried out is the Emtec method, of which one of the main components is the measurement of rubbing noise. However, this method remains a static measurement of softness.

A technique for measuring softness through infrared spectroscopy is proposed in the thesis by Mr Krishan Bhatia presented at the University of Miami, Oxford Ohio en 2004 “USE OF NEAR INFRARED SPECTROSCOPY AND MULTIVARIATE CALIBRATION IN PREDICTING THE PROPERTIES OF TISSUE PAPER MADE OF RECYCLED FIBERS AND VIRGIN PULP”

In this work, an attempt was made to use near infrared spectroscopy combined with chemometric techniques to analyse the results of measurements to predict the properties of softness and tensile strength. Four variables were chosen: nature of the fibres, amount of debonder, amount of wet strength resin and the level of refining. For each of the four variables, spectrum measurements on sheet formers were taken and then the softness and the tensile strength were measured in a conventional manner using the physical methods mentioned above. The method consisted in inferring the softness of the rigidity of a sample, itself measured according to a bending angle of a strip of material, which was held by one end and which was free to bend under its own weight. The spectral absorbance values and data were then used to create a model which was used to predict the properties of unknown samples. The predictions obtained from this study show that it is possible to use NIR spectroscopy combined with sampling and chemometric techniques with multivariate calibration to predict the softness and tensile properties of tissue paper; however, it must be noted that the conditions are those of a laboratory, in static mode, and that softness was correlated with the characteristic of rigidity alone.

### SUMMARY

The NIR spectrometry analysis methods of the prior art are limited to the analysis of physical quantities on the basis of which softness is possibly estimated. Unfortunately, the softness measured in this way is only partial as it takes account of only one of the parameters making up this criterion.

Furthermore, sensory analysis methods have shown themselves to be reliable but difficult to carry out. In an industrial context, they do not provide fast feedback on production variations which could be corrected or adjusted.

Thus, it is desired to develop an automatic method for measuring softness.

It is also desired to develop a method for fast measurement of softness which can be applied in the environment of a paper machine in order to achieve a better control of the paper production.

It is also desired to provide a method which is simple to carry out.

It is also desired to provide a dynamic method which can be carried out during production on the paper machine and which achieves the closest correspondence with the softness measured by a panel.

The method described herein aims to determine the softness of a sheet of tissue paper through NIR spectrometry. According to this method, after having created a model in the form of a database comprising softness values, according to data obtained through NIR spectrometry of a set of reference tissue paper sheets of which the softness is known, the spectral analysis of said sheet is carried out and the softness value thereof is determined on the basis of the model. The method includes obtaining the softness values of said reference tissue paper sheets of the model through sensory analysis.

More particularly, the database of the model, on the basis of which softness is determined, is created by applying chemometric techniques, and more particularly PLS regression.

The sensory analysis can be carried out on a set of 50 to 100 reference tissue paper sheets, the panel then performing the sensory analysis being made up of 10 persons.

The sensory analysis advantageously comprises the taking of a product sample to be evaluated, then the allocation of a grading by comparison with control samples chosen to cover the entire softness range.

The measurement through NIR spectrometry comprises the transmission of a light with a wavelength between 1 and 4 μm.

The method can be applied particularly advantageously to a method for controlling the quality of a sheet of paper during production on a paper machine according to which the softness of the sheet is determined by the method described herein.

The method includes the following steps:
a) Creating a model for a product defined by its range of grammages, its range of thicknesses and its fibre composition on the basis of samples taken from the paper machine.
b) Setting up an NIR spectrum on the sheet during production; the sheet having the same definition, range of grammages, range of thicknesses and fibre composition as the samples of step a).
c) Evaluating the softness of the sheet during production on the basis of the model created in step a).
d) Where appropriate, modifying the operating parameters of the paper machine to improve softness.

In this method, the spectrometric measurements are carried out on the moving sheet present on the paper machine. In certain embodiments, the paper machine includes a Yankee drying cylinder, the spectrometric measurements are carried out downstream of the Yankee and upstream of the winder.

### BRIEF DESCRIPTION OF THE FIGURES

Embodiments of the invention are described below in more detail in the description which follows, with reference to the attached drawings, in which:

FIG. 1 is a diagram showing the optical paths which an incident infrared beam may take and showing the behaviour of the beam in a solid material such as the sheet of tissue.

FIG. 2 is an example of a diagram showing how the measurement is carried out on a sheet of paper.

FIG. 3 shows an example of the graph obtained for the value of the reflectance as a function of the wavelength of the light with which the sheet is illuminated.

FIGS. 4a and 4b are examples showing the correlation obtained between a softness grading given by sensory analysis and the grading obtained on the basis of a prediction model.

### DETAILED DESCRIPTION OF PREFERRED EMBODIMENTS

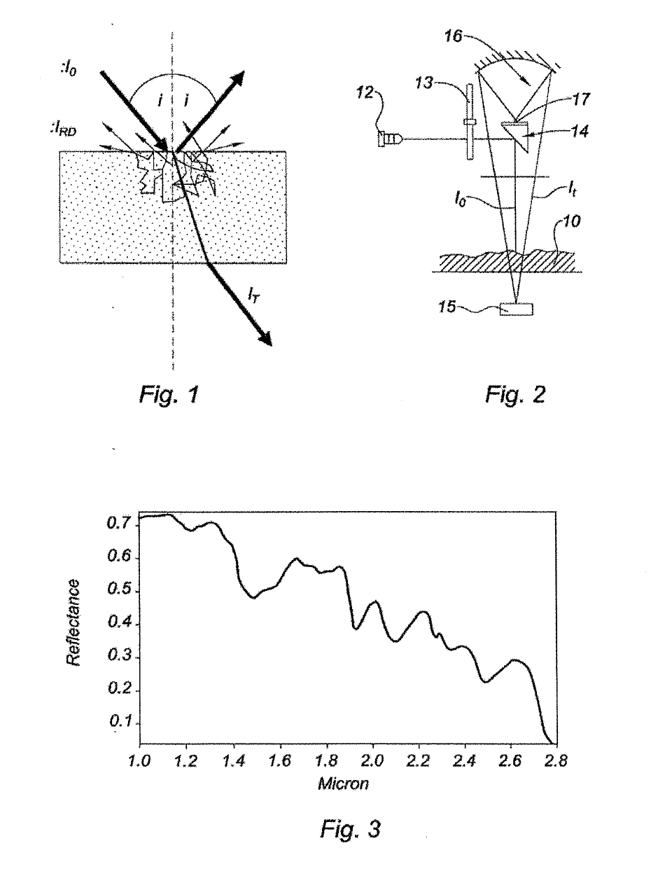

FIG. 1 shows the basic diagram of a device for measurement through conventional infrared absorption. An incident infrared light beam I₀ with a predefined wavelength is directed onto a sample of the material to be analysed, in this case a sheet of paper, then the beam reflected, transmitted Iₜ or diffused, I_{rd}, by the sample is detected by suitable sensors and its intensity is recorded. The signals proportional to the measured light intensity of the detected beam are treated in such a way as to supply a characteristic value of the measured parameter. One or more wavelengths of the transmitted beam are chosen according to their sensitivity to the variation of the considered parameter. Filters are placed between the source and the sample to expose the sample only to the desired extent and to the desired wavelength beam; a detector measures the intensity of the light following interaction with the sample and produces a signal as a function of the intensity of the beam incident on it.

In the simplest case, by calculating the ratio between the signal measured by the detector during the reception of light at the measurement wavelength and the signal of the detector during the reception of light at the reference wavelength, a measurement signal is obtained which gives a measurement of the parameter concerned.

Measurements of a plurality of wavelengths and/or reference wavelengths are can be used, and the signals of measurement wavelengths and reference wavelengths can be used to calculate the parameter concerned.

An example of a measurement device shown in FIG. 2 includes a source of white light 12 focused on a set of filters 13 designed to pass infrared beams with different frequency bands, chosen in the 1-4 μm wavelength range. The light is directed towards a prism mirror 14 which directs the light beam IO towards the cellulose sheet 10. The light beam is partially reflected by the surface of the paper and transmitted for another part across the cellulose sheet before being reflected by a reflector 15. The entire transmitted light signal I_{T} is then collected on a mirror 16 to be directed towards the detector 17.

FIG. 3 shows an example of a graph of the variation in the value of the reflectance I_{T} in relation to the wavelength of the incident light I_{O}.

Once the softness values of a set of tissue paper samples have been obtained through sensory analysis, the sampling step includes correlating said values with the spectral data.

Softness can be a critical parameter in the evaluation of tissue paper products by consumers and can be measured through sensory analysis by panels. The sensory panels includes groups of persons, around ten at least, trained in sensory measurement, whose measurements are monitored in order to ensure their constant accuracy.

The method for measuring softness is designed to be the most representative of the perception of consumers in relation to usage, notably via marketing tests. The method includes evaluating and grading the product to be tested in comparison with reference samples positioned on a defined scale.

For a set of around one hundred products made from tissue, of which the parameters of grammage, fibre quality and possibly additive are known, softness, on the one hand, is measured according to the method described above, then, on the other hand, the reflectance value is measured for predefined wavelengths.

More precisely, a device sold by the company NDC Infrared Engineering was used to carry out the NIR spectrometry measurement.

Once the most appropriate wavelength ranges had been determined, a calibration model was developed through PLS (Partial Least Square) regression.

This statistical tool enables the implementation of the regression of a variable to be explained, i.e. softness, over explanatory variables, i.e. the spectral range data, which may be strongly correlated with one another.

As with multiple linear regression, generally used in statistical analysis, the aim of PLS regression is to construct a linear model of the following type:

Y=XB+E

where B represents the regression coefficients and E the term noise for the model.

Here, Y represents the softness values obtained during the panels and X represents the set of spectral data in the form of numerical data corresponding to the acquisition step, 800 to 1000 measurement points typically being used to represent the reflectance spectrum.

Grading/prediction models were created through cross-validation.

Once the calibration was set up, the method was tested on product samples of which the value and softness were to be predicted.

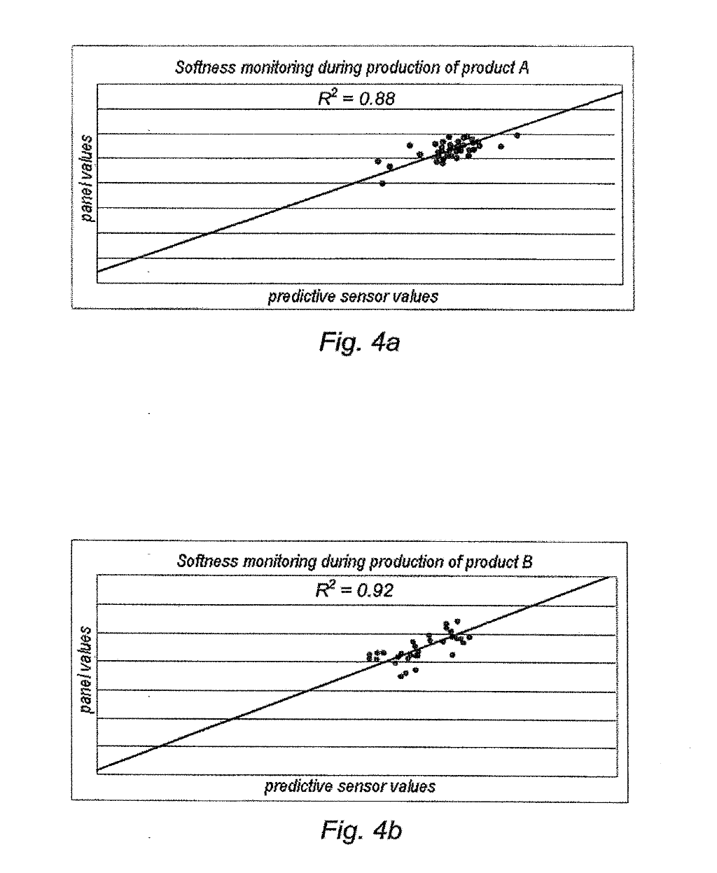

It was noted that excellent results were obtained, as revealed by the graphs in FIGS. 4a and 4b showing the correlation between the values predicted during the production of the tissue sheet and the softness values obtained through sensory analysis. In order to obtain this correlation, samples were taken during production and the collected samples were quantified in terms of their softness, the results being plotted on the basis of measurements carried out through analysis.

The product A corresponds to a strip of tissue paper intended to be transformed into toilet paper.

The physical characteristics are as follows:
Grammage between 15 and 22 g/m²,
Thickness between 1.4 and 2.0 mm for 12 plies,
Fibre composition between 5 and 55% SWK long fibres and between 45 and 95% eucalyptus.

A correlation coefficient R²=0.88 was obtained for this product.

The product B corresponds to a strip of tissue paper intended to be transformed into a pocket handkerchief and a box handkerchief.

The physical characteristics are as follows:
Grammage between 14 and 17 g/m²,
Thickness between 1.2 and 1.085 mm for 12 plies,
Fibre composition between 15 and 30% SWK long fibres and 25 between 70 and 85% eucalyptus.

A correlation coefficient R²=0.86 was obtained for this product.

According to one characteristic, the method is applied during production to the sheet of paper present on the paper machine and provides a production quality control means.

The execution of the method includes:

1) Creating a model for a product defined by its range of grammages, its range of thicknesses and its fibre composition on the basis of samples taken from the paper machine.

2) Setting up an NIR spectrum on a sheet during production; the sheet having the same definition, range of grammages, range of thicknesses, fibrous composition as the sheet of step 1.

3) Evaluating its softness on the basis of the model created in step 1.

4) Where appropriate, modifying the operating parameters of the paper machine to improve softness.

The measurement during production is carried out in a stable position corresponding to the area where the samples are taken for use by the panels, on the basis of which the model is created. A position of this type is located, for example, after the Yankee drying cylinder and before the winder on creped tissue paper. The measurement is carried out, for example, through dynamic “travelling”, in the direction across the width of the strip.

The method as described, thus allows, on the one hand, a softness measurement to be performed on samples in the laboratory for product analysis, said samples originating directly from the paper machine and, on the other hand, a measurement to be performed during production at the output of the paper machine, allowing a control to be carried out during production.

1. A method for determining the softness of a sheet of tissue paper through NIR spectrometry, includes after creating a softness values model according to data obtained through NIR spectrometry for a set of reference tissue paper sheets of which the softness is known, performing a spectral analysis of said sheet and determining a softness value for said sheet the basis of said model, wherein the softness values of said reference tissue paper sheets of the model are obtained through sensory analysis.

2. The method according to claim 1, wherein the model is created by applying chemometric statistical methods.

3. The method according to claim 2, wherein the statistical analysis method is PLS regression.

4. The method according to claim 1, wherein the sensory analysis is carried out on a set of 50 to 100 reference tissue paper samples by a panel made up of 10 persons.

5. The method according to claim 4, wherein the sensory analysis includes taking a tissue paper product sample and then allocating a grading by comparison with reference softness control samples.

6. The method according to claim 1, wherein the measurement through NIR spectrometry comprises the transmission of a light with a wavelength between 1 and 4 μm.

7. A method for controlling the quality of a sheet of paper during production on a paper machine, comprising determining the softness of the sheet by the method according to claim 1.

8. The method according to claim 7, comprising the following steps:
a) creating a model for a product defined by its range of grammages, its range of thicknesses and its fibre composition on the basis of samples taken from the paper machine;
b) setting up an NIR spectrum on the sheet during production; the sheet having the same definition, range of grammages, range of thicknesses and fibre composition as the samples of step a);
c) evaluating the softness of the sheet during production on the basis of the model created in step a); and
d) where appropriate, modifying the operating parameters of the paper machine to improve softness.

9. The method according to claim 8, wherein the spectrometric measurements are carried out on the moving sheet present on the paper machine.

10. The method according to claim 10, wherein the paper machine comprises a Yankee drying cylinder, the spectrometric measurements being carried out downstream of the Yankee and upstream of the winder.
